(19) United States
(12) Patent Application Publication
(10) Pub. No.: US 2004/0146855 A1
(43) Pub. Date: Jul. 29, 2004

(54) Formation of superparamagnetic particles

(76) Inventors: Robert H. Marchessault, Montreal (CA); Kirill Shingel, Montreal (CA); Robert K. Vinson, Montreal (CA); Didier G. Coquoz, Westmount (CA)

Correspondence Address: CLARK & ELBING LLP, 101 FEDERAL STREET, BOSTON, MA, 02110, US

(21) Appl. No.: 10/352,280

(22) Filed: Jan. 27, 2003

(51) Int. Cl. C12Q 1/70; C12Q 1/68; G01N 33/53; G01N 33/567; C04B 35/64; H01F 1/28

(52) U.S. Cl. 435/5; 435/6; 435/7.2; 252/62.53

(57) ABSTRACT

The present invention features a method for preparing superparamagnetic iron particles by the in situ formation of these particles in a cross-linked starch matrix or by the formation of a superparamagnetic chitosan material. The superparamagnetic materials are formed by mild oxidation of ferrous ion, either entrapped into a cross-linked starch matrix or as a chitosan-Fe(II) complex, with the mild oxidizing agent, nitrate, under alkaline conditions. The present invention further features superparamagnetic iron compositions prepared by the method of the invention. The compositions of the invention are useful for the separation, isolation, identification, or purification of biological materials.

### FIELD OF THE INVENTION

The present invention relates to methods for the preparation of superparamagnetic iron particles. The superparamagnetic materials are formed under alkaline conditions by the oxidation of a ferrous ion-polysaccharide matrix with the mild oxidizing agent, nitrate. The present invention further relates to methods for preparing superparamagnetic iron compositions. These compositions may be useful for the separation, isolation, identification, or purification of biological materials.

### BACKGROUND OF THE INVENTION

Magnetic particles are used for a variety of separation, purification, identification, or isolation techniques in connection with biological molecules. Typically, a magnetic particle is coupled to a molecule capable of interacting with another molecule or cell in a biological sample. This interaction can either be specific, e.g., the specific binding between an epitope and a binding region for that epitope, or general, e.g., hydrophobic or ionic interactions. Once a biological sample is brought into contact with the magnetic particle, those biological entities that bind to the magnetic particle are subsequently isolated by application of a magnetic field. Such magnetic separation techniques have been employed to sort cells, to recover antibodies or enzymes from a solution, to purify proteins using affinity techniques, or to remove unwanted particles from a suspension of biological materials. (Ugelstad, et al., “Monodisperse magnetic polymer particles. New biochemical and biomedical applications” Blood Purif., 11(6):349-69 (1993); Setchell, “Magnetic separations in biotechnology—a review” J. Chem. Tech. Biotechnol, 35B:175-82 (1985))

An important feature of magnetic separation is the economy and physicochemical characteristics of the magnetic support. High mechanical resistance and resistance to solvent and microbial attack make inorganic magnetic materials excellent supports, but they lack in functional groups for selective binding of biomolecules of interest. Therefore, inorganic magnetic material is most commonly coated with polymers. Traditionally, the magnetizable particles used for bioseparations have been divided into four general classes:

1. Core-and-shell beads with a magnetic core and a hard shell coating of polymerized monomer or a silanizing agent, e.g., U.S. Pat. No. 4,267,234 (polyglutaraldehyde shell around ferrofluid core particles), U.S. Pat. No. 4,454,234 (suspension or emulsion polymerized coating around submicron magnetic particles), U.S. Pat. Nos. 4,554,088, 4,695,392 and 4,695,393 (silanized magnetic oxide particles of polydisperse size and shape), U.S. Pat. No. 4,672,040 (polysilane coated magnetic particles), U.S. Pat. No. 4,783,336 (suspension polymerized polyacrolein around ferrofluid particles), U.S. Pat. No. 4,795,698 (bovine serum albumin coating), and U.S. Pat. No. 4,964,007 (gelatin-gum arabic-surfactant coating);

2. Core-and-shell beads with a magnetic core and a loose shell of random coil or globular polymer that may or may not be crosslinked, e.g., U.S. Pat. No. 4,452,773 (dextran coating around ferrofluid particles) and U.S. Pat. No. 4,795,698 (protein such as bovine serum albumin around ferrofluid particles;

3. Magnetic latex materials formed by uniformly embedding ferrofluid particles in polystyrene latex particles, e.g., U.S. Pat. No. 4,358,388; and

4. Porous polymer particles filled with magnetic materials, such as polymer-ferrite or polymer maghemite composite systems, Nustad, et al., “Monodisperse Polymer Particles In Immunoassays And Cell Separation”, Microspheres: Medical and Biological Applications, A. Rembaum and Z. Tokes, Eds. (Boca Raton, Fla.: CRC Press, 1988) pages 53-75, C. D. Platsoucas et al., “The Use of Magnetic Monosized Polymer Particles For The Removal Of T Cells From Human Bone Marrow Cell Suspensions”, ibid. at pages 89-99, and U.S. Pat. Nos. 4,563,510, 4,530,956 and 4,654,267.

Magnetically responsive composite microparticles including magnetically responsive materials and porous solid water-insoluble matrices such as proteinaceous materials, polysaccharides and the like, have also been described, e.g., U.S. Pat. No. 4,169,804.

In addition, magnetic cellulose fibers and paper may be prepared by synthesizing ferrites in situ, e.g., U.S. Pat. No. 5,143,583. These fibers have been prepared via careful O₂ oxidation of ferrous hydroxide and precipitated with NaOH from the ferrous ion-exchanged form of the matrix. The chemistry yields magnetic fibers containing small superparamagnetic ferrite (Fe₃O₄) particles of about 10 nm in size. Typically, carboxymethylated cellulose fibers are used as the material subjected to the magnetization scheme, but it has also been suggested that the process could be practiced with a wide range of natural biopolymers.

Lianes, et al., Int. J. of Polymeric Mat. 51:537-45 (2002), disclose superparamagnetic composites of alginate, starch, and chitosan for use in drug delivery or cell sorting, where Fe₂O₃ was formed inside these matrices by oxidation of Fe(II) with hydrogen peroxide.

Particulate ferromagnetic, ferrimagnetic and superparamagnetic agents have also been proposed for use as negative MR contrast agents. Examples of materials which may be used in this way as stabilizers include carbohydrates such as oligo- and polysaccharides, as well as polyamino acids, oligo- and polynucleotides and polyalkylene oxides (including poloxamers and poloxamines), and other materials as proposed in U.S. Pat. Nos. 5,464,696 and 4,904,479.

Given the many uses of matrices of superparamagnetic particles that interact with biological materials and, particularly, those matrices that are polysaccharide-based, there remains a need in the art for a method of preparing superparamagnetic particles where Fe(II) is oxidized in the presence of a polysaccharide-based matrix under mild and efficient conditions. Polysaccharide matrices are particularly desirable for in vitro or in vivo use as they typically do interact non-specifically with proteins (e.g., they do not have attractive hydrophobic interactions with proteins). Ideally, the method of iron oxidation employed in a polysaccharide-based matrix to yield a superparamagnetic particle or composition should proceed in high yield with minimal or no oxidative degradation of the matrix. Additionally, it is desirable that the oxidation be performed in situ, so that the process is scalable and easy to control. It is also desirable that this method produces particles or compositions useful for the separation, isolation, identification, or purification of biological materials.

### SUMMARY OF THE INVENTION

In a first aspect, the invention features a method for the in situ formation of superparamagnetic particles in a polysaccharide matrix where an Fe(II) salt is diffused into a starch matrix, thereby entrapping Fe(II) ions within the matrix, and where the Fe(II) ions are oxidized with nitrate under alkaline conditions, converting the starch matrix of Fe(II) ions into superparamagnetic ferric oxide particles.

In an embodiment of the first aspect, the polysaccharide matrix can be starch, cross-linked starch, chitosan, chitin crystallites, dextran, cross-linked dextran, cellulose, cellulose fibers, microcrystalline cellulose, alginic acid, hyaluronic acid, glycogen, or a glycosylaminoglycan. Desirably, the polysaccharide matrix can be cross-linked starch, chitosan, chitin crystallites, cross-linked dextran, cellulose fibers, or microcrystalline cellulose, and most desirably, the polysaccharide matrix can be cross-linked starch or chitosan.

In another desirable embodiment of the invention, alkaline conditions are provided by contacting the matrix with ammonium hydroxide. Additional embodiments include the use of sodium nitrate, potassium nitrate, cesium nitrate, ammonium nitrate, tetra(C₁-C₈ alkyl)ammonium nitrate, or barium nitrate as the oxidant, with the most desirable oxidant being sodium nitrate. It is also most desirable to use FeCl₂ as the source of FE(II) ions.

In a second aspect, the invention features a method for the in situ oxidation of a superparamagnetic particle polysaccharide matrix which includes the steps of, a) maintaining a solution or a suspension of said superparamagnetic iron particle at a pH of about 7.5 to about 10.5 by the addition of a base, b) maintaining the basic solution/suspension of said superparamagnetic iron particle at a temperature of about 0° C. to about 20° C., c) adding sodium bromide and a catalytic amount of TEMPO (2,2,6,6-tetramethyl-1-piperidinyloxy, free radical) to the solution/suspension, and d) adding sodium hypochlorite to the solution/suspension. As an embodiment of the second aspect, sodium chlorite may be added as an additional oxidant after the addition of TEMPO to the reaction mixture. In an additional embodiment, the superparamagnetic particle that is oxidized is one that is prepared by the method of the first aspect of the invention.

In a third aspect, the invention features a composition prepared by the method of the second aspect of the invention, having a starch-based matrix and a superparamagnetic iron oxide particle within this matrix, wherein the matrix is about 5 percent to about 30 mole percent carboxyl groups.

In a fourth aspect, the invention features a composition formed using the method of the first or second aspect, wherein the composition includes a polysaccharide matrix and a superparamagnetic iron oxide particle within the polysacchanide matrix. This composition may also include a second biological molecule different than the polysaccharide included in the matrix. Desirably, the second biological molecule is covalently attached to the polysaccharide matrix.

In a fifth aspect, the invention features a method for the separation, isolation, identification, or purification of a biological entity using a composition of the invention that includes contacting the composition with the biological entity and affecting the separation, isolation, identification, or purification with a magnetic field. In an embodiment of this aspect, the composition includes a biological molecule that interacts positively with the biological entity. The biological entity can include a cell, a virus, or a phage. Desirably, the biological entity includes a protein, a peptide, a carbohydrate, a glycopeptide, a glycoprotein, a glycosylaminoglycan, a cationic lipid, a glycolipid, or a polynucleotide. Most desirably, the biological entity includes a protein.

By the term “affinity ligand” is meant a moiety that binds selectively or preferentially to a component of the target material to be isolated, purified, separated, or analyzed through a specific interaction with a binding site of the component. In the practice of the present invention, the affinity ligand is typically associated with the superparamagnetic particle or composition prepared by the method of the invention. Examples of affinity ligands that may be useful in the method of the present invention include: protein A and protein A analogs, which selectively bind to immunoglobulins; dyes; antigens, useful for purification of associated antibodies; antibodies, for purification of antigens; substrates or substrate analogs, for purification of enzymes; complementary polynucleotides; and the like.

In the present context, the term “alkaline conditions” refers to conditions where the pH of a solution is greater than neutrality, i.e., pH>7.0. These conditions can be achieved by the addition of an organic or inorganic base to an aqueous solution or to a mixed aqueous/organic solution.

By “biological entity” is meant a substance that is naturally occurring, derived from a substance that is naturally occurring, or an analog of a substance that is naturally occurring. Biological entities can include cells, viruses, phages, and the like. Biological entities can also include biological molecules, as defined below.

By “biological molecule” is meant a substance that contains naturally occurring units, subunits, or analogues thereof. Biological molecules can be, but are not restricted to, proteins, peptides, carbohydrates, glycopeptides, glycoproteins, glycosylaminoglycans, cationic lipids, glycolipids, or polynucleotides. In addition, biological molecules may be synthetic molecules containing unnatural amino acids, unnatural nucleotides, and the like. Biological molecules may also be those entities derived from recombinant technology.

By “carbohydrate” is meant any group of organic compounds based on the general formula Cₓ(H₂O)_{y}, or a derivative thereof. Carbohydrates include monosaccharides, oligosaccharides, and polysaccharides. Carbohydrates may also vary from this general formula and include deoxy-compounds, such as 2-deoxy-D-ribose, where one or more hydroxy groups of the carbohydrate are replaced by hydrogen.

By “Contramid®” is meant the proprietary excipient of Labopharm, Inc., based on modified high amylose starch.

By “diffusing” is meant the dissemination of a substance in a matrix. Diffusing is usually accomplished by mixing or by waiting a sufficient time period after a substance comes into contact with a matrix material for the substance to be evenly distributed within the matrix.

By “entrapment” is meant the binding of a substance in a matrix. Binding may be either through covalent or non-covalent interactions.

By “glycosylaminoglycan” is meant a carbohydrate wherein one or more than one hydroxyl groups are replaced with amino groups or derivatized amino groups (e.g., N-acetyl groups).

The phrase “interacts positively” is meant to describe any contact between two entities, typically two different biological entities, which results in a thermodynamically favorable interaction. Typically, this interaction is between an affinity ligand and its biological cognate. Other examples of positive interactions are thermodynamically favorable hydrophobic interactions or thermodynamically favorable ionic interactions.

By “lipid” is meant any member of fatlike substances that occur in living organisms. The term lipid may include: fatty acids, triacylglycerols or other fatty acid esters, long chain alcohols and waxes, sphingoids or other long-chain bases, glycolipids, phospholipids, sphingolipids, carotenes, polyprenols, sterols, and terpenes. The term “cationic lipid” refers to a lipid with one or more positive charges. The term “glycolipid” refers to a lipid that is covalently bound to a carbohydrate.

By “MagCon” is meant high amylose crosslinked starch that has been converted to the superparamagnetic state.

By “MagConC” is meant MagCon that has been prepared from Contramid®.

By “MagChi” is meant chitosan that has been converted to the superparamagnetic state.

By “matrix” is meant a binding substance or material. Binding may be either through covalent or non-covalent interactions. In general, the matrices of the invention are polymeric, or mixtures of polymeric functionalized organic compounds. Exemplary matrices are starch, chitosan, or other polysaccharides.

By “monosaccharides” are meant polyhydric alcohols from three to ten or more carbon atoms containing either and aldehyde group (e.g., aldoses) or a keto group (e.g., ketoses), or masked aldehyde or keto groups, or derivatives thereof. Examples of monosaccharide units are the D and L configurations of glyceraldehyde, erythrose, threose, ribose, arabinose, xylose, lyxose, allose, altrose, glucose, mannose, gulose, idose, galactose, talose, dihydroxyacetone, erythrulose, ribulose, xylulose, puscose, fructose, sorbose and/or tagatose. Examples of monosaccharides also include those monosaccharide deoxy sugars, such as, for example, fucose, rhamnose, and digitoxose; deoxyamino sugars such as, for example, glucosamine, mannosamine, galactosamine; deoxyacylamino sugars such as, for example, N-acetylglucosamine, N-acetylmannosamine, and N-acetylgalactosamine; and aldonic, aldaric and/or uronic acids such as, for example, gluconic acid or glucuronic acid. Monosaccharides also include ascorbic acid, amino acid-carrying monosaccharides and monosaccharides which carry lipid, phosphatidyl or polyol residues.

By “polynucleotide” is meant a homo- or heteropolymer of two or more nucleotide units connected by phosphodiester linkages.

The term “particles” as used herein encompasses spheres, spheroids, beads and other shapes as well. In addition, particles with the magnetic properties of superparamagnetism, fenimagnetism and ferromagnetism are referred to herein as “magnetic particles”. In the present invention, the term particles can refer to superparamagnetic starch particles, superparamagnetic, chitosan particles, or any other superparamagnetic particle with a polysaccharide matrix.

By “peptide” is meant a molecule that contains from 2 to 100 natural or unnatural amino acid residues joined by amide bonds formed between a carboxyl group of one amino acid and an amino group from the next one. The term “glycopeptide” refers to a peptide that is covalently bound to a carbohydrate.

The term “polysaccharide” is meant to include any polymer of monosaccharides, or salts therein, and includes disaccharides, oligosaccharides, etc. Polysaccharides include starch, dextran, cellulose, chitosan, glycogen, hyaluronic acid, alginic acid, and glycosylaminoglycans. The polysaccharide of this invention may be unmodified or modified and the term polysaccharide is used herein to include both types. By modified polysaccharide it is meant that the polysaccharide can be derivatized or modified by typical processes known in the art, e.g., esterification, etherification, oxidation, acid hydrolysis, cross-linking and/or enzyme conversion. Typically, modified polysaccharides include esters such as the acetate and the half-esters of dicarboxylic acids, particularly the alkenylsuccinic acids; ethers, such as hydroxyethyl and hydroxypropyl starches and starches reacted with hydrophobic cationic epoxides; starches oxidized with hypochlorite; starches reacted with cross-linking agents such as phosphorous oxychloride, epichlorohydrin or phosphate derivatives prepared by reaction with sodium or potassium orthophosphate or tripolyphosphate and combinations thereof. These and other conventional modifications of starch are described in publications such as Starch: Chemistry and Technology, 2nd Edition, Ed. Whistler, BeMiller, and Paschall, Academic Press, 1984, Chapter X.

By “protein” is meant a molecule that contains over 100 natural or unnatural amino acid residues joined by amide bond(s) formed from a carboxyl group of one amino acid and an amino group from the next one. The term “glycoprotein” refers to a protein that is covalently bound to a carbohydrate.

By the term “superparamagnetic” is meant a material that is highly magnetically susceptible, i.e., it becomes strongly magnetic when placed in a magnetic field, but like a paramagnetic material, rapidly loses its magnetism and displays no remanence once the magnetic field has been removed.

### BRIEF DESCRIPTION OF THE DRAWINGS

FIG. 1 is a reaction scheme outlining the results from the treatment of Fe(OH)₂ with various oxidants.

FIG. 2 is a composite of solid-state ¹³C-NMR spectra of Contramid® (a) and carboxylated MagCon samples (b, c).

FIG. 3 is a composite of FTIR spectra from Contramid® (a), MagCon prepared by nitrate oxidation (b), and MagCon prepared by peroxide oxidation (c).

FIG. 4 is a composite of FTIR spectra of chitosan (a), MagCon-COOH (b), MagCon-COOH conjugated with chitosan, 1:0.3 (c), and MagCon-COOH conjugated with chitosan, 1:1 (d).

FIG. 5 is a composite of FTIR spectra of MagCon conjugated to bovine serum albumin (BSA) at BSA reaction mixture concentrations of 0 mg/mL, 2 mg/mL, 10 mg/mL, and 50 mg/mL.

FIG. 6 is a composite of FTIR spectra of MagCon-COOH and MagCon-COOH conjugated to bovine serum albumin (BSA) at BSA reaction mixture concentrations of 0 mg/mL, 1.6 mg/mL, 8 mg/mL, and 40 mg/mL.

### DETAILED DESCRIPTION OF THE INVENTION

The invention provides a method for the in situ formation of superparamagnetic particles in a cross-linked starch matrix. The method involves: (a) diffusion an Fe(II) salt into a polysaccharide matrix, thereby entrapping Fe(II) ions within the matrix and, (b) oxidizing the Fe(II) ions with nitrate under alkaline conditions, converting the polysaccharide matrix of Fe(II) ions into superparamagnetic ferric oxide particles. Examples of polysaccharide matrices used for the formation of superparamagnetic particles are cross-linked starch, chitosan, chitin crystallites, Sephadex™ (dextran beads cross-linked with epichlorohydrin), cellulose fibers, and Avicel® (microcrystalline cellulose), the properties of which are shown in Table 1.

TABLE 1
Examples of polysaccharides used for in situ synthesis of
superparamagnetic particles.

| | | Functional |
|---|---|---|
| Name | Description | Group |
| Cross-linked | Epichlorohydrin cross-linked amylose, | —OH, can be |
| starch | Hylon-VII, amylose content ˜70% | transformed to |
| | | —COOH |
| Chitosan | A copolymer of β-(1-4)-linked 2-amino- | —NH₂, —OH |
| | 2-deoxy-D-glucose, readily prepared | |
| | from chitin by chemical N-deacetylation | |
| Chitin | Rod-like crystallites of chitin obtained | —NH₂, —OH |
| crystallites | via acid hydrolysis; size: about 1 μm | |
| Sephadex | Epichlorohydrin cross-linked dextran | —OH |
| | beads; stable at pH 3-12; size: 20-40 μm | |
| CLD fibres | Cellulose fibres; stable at pH 3-12 | —CH₂COOH |
| | | and —OH |
| Avicel | Microcrystalline cellulose (Avicel PH- | —OH, can be |
| | 102 and PH-103); insoluble in water, | transformed to |
| | dilute acids, and most organic solvents; | —COOH |
| | practically insoluble in dilute NaOH | |
| | solution; size: 90-180 μm | |

Due to its favorable properties and its ready availability, cross-linked starch is a desirable polysaccharide of the present invention. The chemistry of crosslinking polysaccharides is well known and there are a variety of agents to crosslink hydroxyl groups of polysaccharides. In the present context cross-linked starch is prepared by treating granular starch with multifunctional reagents capable of forming linkages with hydroxyl groups in the starch (Park, et al., “Crosslinking of water-soluble polymers” in Biodegradable Hydrogels for Drug Delivery, Technomic Publishing Co., USA, 1993: pgs 73-82). The two components of starch granules, amylose and amylopectin, can vary in relative amount, e.g., from about 2 percent to about 90 percent amylose. Cross-linking of amylose can be carried out in the manner described by Mateescu in Biochimie 60:535-37 (1978) by reacting amylose with epichlorohydrin in an alkaline medium. In the same manner, amylose can also be cross-linked with other cross-linking agents including, but not limited to, 2,3-dibromopropanol, epichlorohydrin, sodium trimetaphosphate, linear mixed anhydrides of acetic and di- or tribasic carboxylic acids, vinyl sulfone, diepoxides, cyanuric chloride, hexahydro-1,3,5-trisacryloyltriazine, hexamethylene diisocyanate, toluene 2,4-diisocyanate, N, N-methylenebisacrylamide, N,N′-bis (hydroxymethyl)ethyleneurea, phosgene, tripolyphosphate, mixed carbonic-carboxylic acid anhydrides, imidazolides of carbonic and polybasic carboxylic acids, imidazolium salts of polybasic carboxylic acids, guanidine derivatives of polycarboxylic acids, and esters of propanoic acid. In a desirable embodiment of the invention, the amylose content is above 40%. In a most desirable embodiment, the amylose content is above 60%. Commercially available Contramid® has an amylose content of about 70%.

Another desirable polysaccharide of the present invention is chitosan, a linear copolymer of β-(1-4)-linked 2-amino-2-deoxy-D-glucose, which is readily prepared from chitin by chemical N-deacetylation. In chitosan, generally about 80% of the units are deacetylated, with the remaining 20% acetylated. These values can vary with chitin sources and with processing methods. The chemical and biochemical reactivity of chitosan is higher than that of chitin because chitosan has free primary amino groups distributed regularly in its chain. Therefore, chitosan is soluble by salt formation because the primary amines can be protonated by certain selected acids (Muzarelli, et al., Structural and functional versatility of chitins” in Polysaccharides. Structural, diversity, and functional versatility, Severian Dumitriu, Ed., Marcel Dekker, Inc. 1998, pgs. 575-77).

When the Fe(II) salt is brought into contact with the polysaccharide matrix, the salt is diffused in the matrix before the nitrate oxidation step. This is normally accomplished, after its addition, by mixing the mixture by any number of means, including shaking, swirling, mechanical stirring, or magnetic stirring. Most desirably, the mixture is mechanically stirred.

Examples agents suitable for providing the alkaline conditions for the first aspect of the present invention include, but are not limited to, sodium carbonate, potassium carbonate, tetraalkylammonium hydroxides, and ammonium hydroxide. Desirably, alkaline conditions are provided by contacting the matrix with ammonium hydroxide. Desirably, the pH of the reaction mixture after the addition of base is from 10 to 14. Most desirably, the pH is 12.

The source of Fe(II) can be from one or a combination of different Fe(II) salts or complexes. These sources include Fe(OAc)₂, (NH₄)₂Fe(SO₄)₂, FeBr₂, FeCl₂, FeC₂O₄ (iron oxylate), FeSO₄, or Fe(ClO₄)₂. In desirable embodiment of the first aspect of the present invention, the source of Fe(II) ions includes FeCl₂, Fe(OAc)₂, or (NH₄)₂Fe(SO₄)₂. Most desirably, FeCl₂ is used as the source of Fe(II) ions.

The oxidizing step is performed under alkaline conditions in such a manner that Fe(II) is oxidized to Fe(III). Desirably, the product of iron oxidation is the superparamagnetic species Fe₃O₄ (i.e., magnetite). Oxidants that can be used for this purpose include sodium nitrate, potassium nitrate, cesium nitrate, ammonium nitrate, tetraalkylammonium nitrate, wherein alkyl is linear or branched C₁-C₈ alkyl, silver nitrate, or barium nitrate. Most desirable is when potassium nitrate is the source of nitrate.

The choice of the polysaccharide ultimately used to form a superparamagnetic particle of the invention may depend on the functional groups (e.g., hydroxyl, amine, carboxyl) that are contained within. Superparamagnetic particles or compositions that result from the further manipulation of these functional groups include the attachment of ligands or other functional moieties. Functional group manipulation can occur either before or after Fe(II) oxidation.

As an example of functional group manipulation, a third aspect of the present invention features a method for the in situ oxidation of a polysaccharide matrix that includes a superparamagnetic iron particle to produce a carboxylated superparamagnetic particle or composition. An embodiment of this aspect includes superparamagnetic particles prepared by the method of the first aspect of the present invention. One method for the in situ oxidation of a polysaccharide matrix includes maintaining a solution or suspension of at a pH of between about 7.5 to about 10.5, desirably a pH of between about 9 to about 10, most desirably a pH of about 9.5, by the addition of a base. Exemplary bases are carbonate or hydroxide. Most desirably, the base used is ammonium hydroxide. The method also includes maintaining the basic solution/suspension of the superparamagnetic particle at a temperature of between about 0° C. and 20° C., desirably between about 0° C. and 10° C., most desirably between about 2° C. and 5° C. The method includes adding sodium bromide and a catalytic amount of TEMPO (2,2,6,6-tetramethyl-1-piperidinyloxy, free radical) to the solution/suspension, followed by the addition of sodium hypochlorite. Desirably, the amount of TEMPO added is between about 2 to 7 mole percent of the mole equivalents of COOH groups desired with the amount of sodium hypochlorite used stoichiometric to the number of mole equivalents of COOH groups desired. In an embodiment of this aspect, sodium chlorite is also added to the solution suspension with the addition of sodium hypochlorite. Desirably, the sodium chlorite added is stoichiometric to, and the amount of sodium hypochlorite added is about 3 mole percent of, that of the number of mole equivalents of COOH groups desired. A procedure for the use of sodium chlorite in TEMPO-mediated oxidations can be found in Zhao, et al., J. Org. Chem. 64:2564-6 (1999).

Another aspect of the invention features a composition that includes a superparamagnetic particle formed by the in situ nitrate oxidation of Fe(II) in a polysaccharide matrix. Yet another aspect of the invention features a superparamagnetic particle composition formed from the in situ oxidation of Fe(II) in a polysaccharide matrix that includes carboxyl groups. In the latter aspect, the superparamagnetic iron particle may have been formed by nitrate oxidation of Fe(II). Preferably, a composition formed from the in situ oxidation of Fe(II) in a starch matrix has from about 5% to about 30% mole percent carboxyl groups. A composition of the invention may also include pharmaceutically unobjectionable excipients and/or auxiliary substances. In some embodiments, a composition of the invention includes a second biological molecule other than the polysaccharide matrix. In one embodiment, the second biological molecule is covalently bound to the polysaccharide matrix. If the polysaccharide matrix contains a reducing sugar, a particularly useful method of attaching a second biological molecule is by a reductive amination procedure, reacting the carbonyl of the reducing sugar with an amine of the second biological molecule, followed by reduction of the resulting imine to an amine with cyanoborohydride. If the polysaccharide contains carboxylic acids, a useful method of attaching a second biological molecule is by forming amides between the polysaccharide and amines present on the second biological molecule. This can be done using standard amide bond forming reagents, desirably by the use of 1-[3-(dimethylamino)propyl]-3-ethylcarbodiimide (EDCI). If the polysaccharide contains an amine, useful ways of attaching a second biological molecule include reacting the amine on the polysaccharide with either a carbonyl or a carboxyl functionality on the second biological molecules in manners similar to those described above.

In a desirable embodiment of the invention, the composition includes affinity ligands, such as antibodies or antibody fragments, antigens, biotin, steptavidin, enzyme substrates or substrate analogs, protein A or protein A analogs, or complementary polynucleotides. These affinity ligands or other biological materials can be attached to the matrix after Fe(II) oxidation or, preferably, be included in the matrix before nitrate-mediated oxidation of Fe(II). Affinity ligands and methods of binding them to support materials are well known in the purification art, e.g., the reference texts Affinity Separations. A Practical Approach (Practical Approach Series), Matejtschuk (Editor), Irl Pr: 1997 and Affinity Chromatography, Herbert Schott, Marcel Dekker, New York: 1997.

In another aspect, a composition of the invention may be used for separation, isolation, identification, or purification of biological entities. In one embodiment, the composition is brought into contact with a biological entity that interacts positively with an affinity ligand of the composition. Purification of the biological entity is then affected by the application of a magnetic field or gradient, such as those used in high gradient magnetic separations (HGMS). In a desirable embodiment, the biological entity can be a cell, a virus, or a phage. For example, a composition of the invention can be used for cell separation. Typically, the composition is brought into contact with the cell or cells and a complex is allowed to form between the composition and the cells. A magnetic field is then used to isolate the complex using an appropriate magnetic separator.

In another desirable embodiment of the invention, the biological entity can include a protein, a peptide, a carbohydrate, a glycopeptide, a glycoprotein, a glycosylaminoglycan, a cationic lipid, a glycolipid, or a polynucleotide. In a most desirable embodiment, the biological entity is a protein.

Another use of a composition of the invention is in the synthesis of polymeric biological molecules (e.g., peptides, polynucleotides). In this method, stepwise synthesis is performed using a composition of the invention as the solid support. Thus, attachment of one monomeric building block and repetitive addition of subsequent monomeric building blocks (e.g., amino acid derivatives, nucleotide derivatives) to the composition can be combined with magnetic separation at appropriate times in the synthesis for the removal of reaction by-products. Compositions of the present invention can also be used as a solid support for combinatorial chemistry.

A composition of the invention may also be especially valuable for use as in vivo diagnostic agents. Particularly desirable is the use of these compositions as NMR contrast agents. Typically, the composition, in a pharmaceutically acceptable carrier, is administered to a patient, orally, intraperitoneally, or intravascularly, followed by subjecting the patient to NMR imaging.

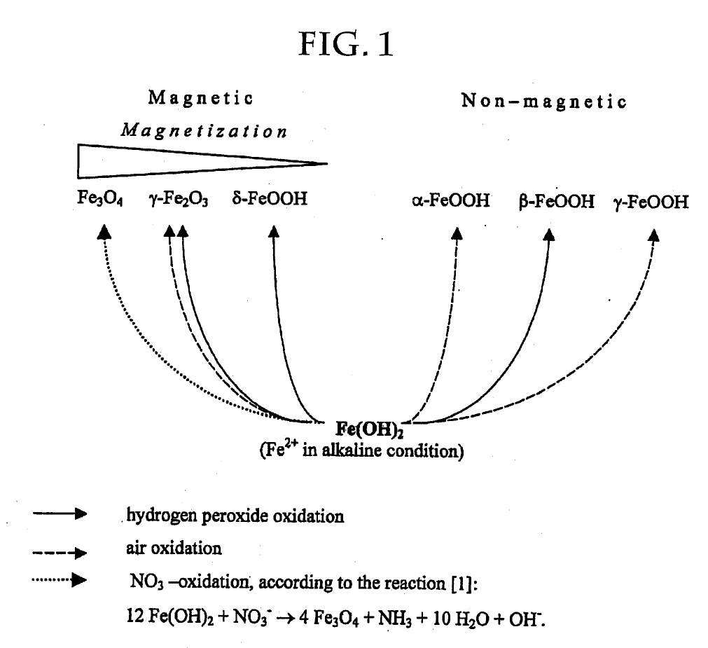

Several methods can be used in the analysis of the superparamagnetic particles and compositions of the invention. Iron content can be determined by atomic absorption spectroscopy (AAS) or by extraction sample magnetometry (ESM) measurements. In ESM, about 20 mg of magnetic materials at room temperature are vibrated in a magnetic field varying from −1.5 to 1.5 T. The data of magnetization as a function of the applied field were recalculated as the percents of iron in the magnetic particles. In these calculations, the standard values of magnetization of magnetite (84-90 J/T kg) were employed. As shown in FIG. 1, other oxidation methods either produce non-magnetic iron species (e.g., α-FeOOH, β-FeOOH, or γ-FeOOH) or species with a reduced magnetization properties, such as γ-Fe₂O₃ or δ-FeOOH. As a result of this, in those cases where iron species other than Fe₃O₄ are produced, it is expected that iron content determined via extraction sample magnetometry be lower than iron content determined by atomic absorption spectroscopy. This is generally observed, as shown in Tables 2-4, if one compares the ESM and AAS values for the formation of superparamagnetic particles synthesized via nitrate oxidation and those prepared via peroxide oxidation. In addition to differences in the iron content values, the product yields obtained from particles via nitrate oxidation are higher. This may be due to the relative lack of polysaccbaride matrix decomposition observed when nitrate is used as the oxidant.

TABLE 2
Characteristics of cross-linked superparamagnetic
particles synthesized via peroxide oxidation (PX).

| | Starting | | | Oxidation | | |
|---|---|---|---|---|---|---|
| | Material | Product | Yield | Cycles | Iron Content (%)² | |
| Sample¹ | (g) | (g) | (%) | (No.) | ESM | AAS |
| MagConI (PX) | 1.0 | 0.32 | 32.0 | 3 | 36.1 | 33.3 |
| MagConII (PX) | 3.5 | 1.62 | 46.3 | 3 | 10.3 | 28.8 |
| MagCon | 2.0 | 0.43 | 21.5 | 3 | 13.8 | 23.3 |
| 8.5COOH (PX) | | | | | | |
| MagCon | 3.0 | 0.33 | 11.0 | 3 | 22.0 | 25.3 |
| 34COOH (PX) | | | | | | |
| MagChi (PX) | 5.0 | 4.92 | 98.4 | 3 | 22.6 | 46.4 |

¹I & II refer to experiment numbers, 8.5COOH and 34COOH refers to degree of carboxylation (i.e., 8.5% and 34%)
²ESM is extraction sample magnetometry, AAS is atomic absorbance spectroscopy

TABLE 3
Characteristics of cross-linked superparamagnetic
particles synthesized via nitrate oxidations.

| | Starting | | | Oxidation | | |
|---|---|---|---|---|---|---|
| | Material | Product | Yield | Cycles | Iron Content (%)² | |
| Sample¹ | (g) | (g) | (%) | (No.) | ESM | AAS |
| MagConI | 5.0 | 3.91 | 78.2 | 1 | 6.8 | 6.2 |
| MagConII | 5.0 | 4.86 | 97.2 | 1 | 10.1 | 11.44 |
| MagCon | 5.0 | 4.56 | 91.2 | 1 | 16.8 | 11.78 |
| 30COOH | | | | | | |
| MagConCI | 2.0 | 2.03 | 102.0 | 1 | n/d | 39.6 |
| MagConCII | 2.0 | 2.20 | 110 | 1 | n/d | 35.4 |
| MagCon | 5.0 | 4.3 | 86.5 | 1 | 62.7 | 46.0 |
| 20COOH | | | | | | |

¹I & II refer to experiment numbers, 30COOH and 20COOH refers to degree of carboxylation (i.e., 30% and 20%), MagConC is derived from commercially available Contramid ®.
²ESM is extraction sample magnetometry, AAS is atomic absorbance spectroscopy

TABLE 4
Characteristics of SPMPs prepared by oxidizing Fe(II)
with nitrate using various polysaccharide matrices.

| | Starting | | | Oxidation | Iron | |
|---|---|---|---|---|---|---|
| | Material | Product | Yield | Cycles | Content (%)² | |
| Sample¹ | (g) | (g) | (%) | (No.) | ESM | AAS |
| MagChi | 5.00 | 14.90 | 300 | 1 | 62-68 | 42.54 |
| Magnetic | 2.00 | 1.86 | 93 | 1 | 14-15 | 9.02 |
| Sephadex | | | | | | |
| Magnetic | 5.00 | 1.96 | 98 | 1 | 8-10 | 6.08 |
| Avicel I | | | | | | |
| Magnetic | 3.50 | 5.3 | 106 | 1 | n/d | 5.70 |
| Avicel II | | | | | | |
| Magnetic CLD | 3.00 | 0.75 | 100 | 1 | 31-34 | 24.5 |
| fibers | | | | | | |
| Magnetic Chitin | 3.00 | 3.1 | 103 | 1 | n/d | 18.07 |
| crystallites | | | | | | |

¹I & II refer to experiment numbers 2 ESM is extraction sample magnetometry, AAS is atomic absorbance spectroscopy

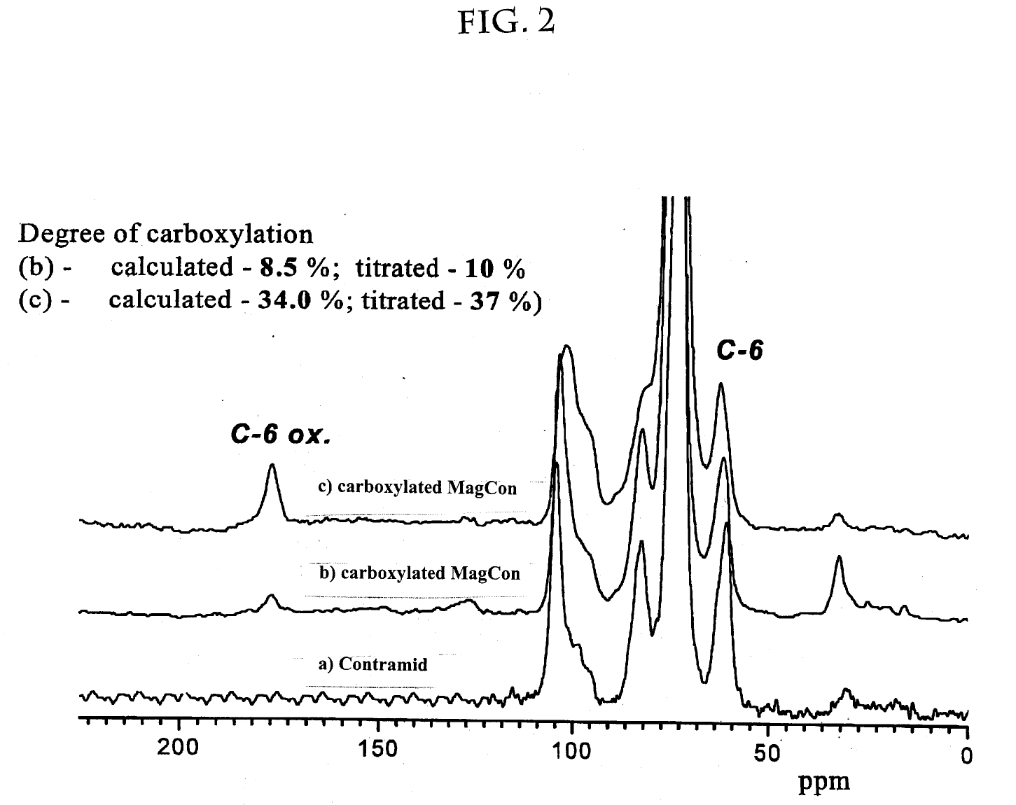

Solid-state ¹³C-NMR spectra of MagCon and its carboxylated derivatives were recorded on a Chemagnetics CMX-300 spectrometer. Integral intensities of the signals at 62 ppm (C-6 hydroxymethyl) and 178 ppm (C-6 carboxyl) were used for determination of the degree of carboxylation (D.C.): D.C. (%)=[C-6 carboxyl/(C-6 hydroxymethyl+C-6 carboxyl)]×100. Shown in FIG. 2 is a composite of solid-state ¹³C-NMR spectra of Contramid® (a) and carboxylated MagCon samples (b, c).

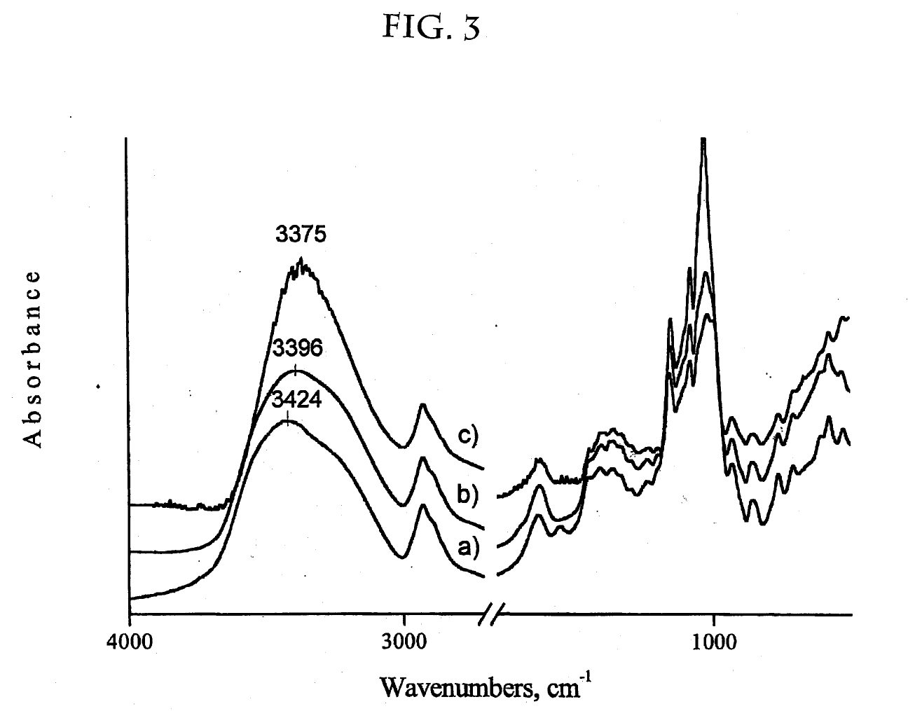

Fourier-transform infrared spectra (FTIR) as an average of 100 scans with a 4 cm⁻¹ resolution were recorded with a Brüker IFS 48 spectrometer. The samples (0.03 g) were prepared in the form of a pellet in KBr (0.2 g). Further evidence of differences between superparamagnetic MagCon particles obtained via nitrate oxidation and peroxide oxidation can be found by observing their FTIR spectra. FIG. 3 shows normalized spectra of Contramid®, MagCon produced by nitrate oxidation (MagCon NT), and MagCon produced by peroxide oxidation (MagCon PX). The material produced by the peroxide oxidation results in an OH-stretch band at about 3375 cm⁻¹ that has greater intensity and is shifted more towards lower wavenumbers than particles obtained by nitrate oxidation. This is believed to be due to the presence of unreacted Fe(OH)₂ (see Ruan, et al., Spectrochim. Acta A, 57:2575-86 (2001)) or other hydrated iron species, such as β-FeOOH, in the particles obtained via peroxide oxidation.

The following non-limiting examples are illustrative of the invention.

### EXAMPLE 1

### In Situ Synthesis of Superparamagnetic Cross-Linked Starch Particles (MagCon) by the Nitrate Mediated Oxidation of Fe(II) Ions

A suspension of 5 g of Contramide (a high amylose cross-linked starch) in 100 mL of fresh deionised water was added to 250 mL of an aqueous solution of 0.5 M FeCl₂. The suspension was stirred under reduced pressure, thereby removing all gases from the suspension and also facilitating diffusion of Fe ions into the porous Contramid® matrix. After 30 min of stirring, the swollen beads of the Contramid®-Fe complex were separated by centrifugation and washed several times with deionised water. The resulting Contramid®-Fe particles were re-suspended in 250 mL of deionised water and 200 mL of 0.5 M NH₄OH was added, turning the mixture dark green. Immediately after NH₄OH addition, the mixture was placed into a water bath kept at 70°-80° C. and 30 mL of 10% (w/w) KNO₃ was added. The reaction mixture was stirred at this temperature for 60 min. Nitrate oxidizes Fe(II) to Fe₃O₄ according to the following formula:

12Fe(OH)₂+NO₃⁻→4Fe₃O₄+NH₃+10H₂O+OH⁻

After 60 min., the flask was removed from the water bath and the reaction mixture was stirred for another 10 min. The resulting dark grey particles (MagCon particles) were collected by centrifugation, washed with water, washed with 0.1 M acetic acid, and lyophilized. The final product yield typically ranges from 3.9 g to 5.0 g, which corresponds to a recovery yield of 78 to 97%, and contains up to 50% (w/w) of iron in the form of Fe₃O₄, as determined by atomic absorbance spectroscopy (AAS) or extraction sample magnetometry (ESM), as shown in Table 3.

### EXAMPLE 2

### In Situ Formation of the Superparamagnetic Chitosan Particles (MagChi) by Nitrate-Mediated Oxidation of Iron (II) Ions

Chitosan (5 g) was dissolved in 100 mL of 0.1 M acetic acid to give a viscous chitosan solution. This solution was transferred into the flask containing 25 g of FeCl₂ in 500 mL of water and the mixture obtained was stirred under reduced pressure for 30-50 min. After incubation, the chitosan-Fe complex was precipitated by the addition of 200 mL of 0.5 M NH₄OH and the resultant dark-green gel was broken up by intense stirring and washing several times with dejonised water. The resulting chitosan-Fe(OH)₂ particles were resuspended in 200 mL of deionised water and 400 mL of 0.5 M NH₄OH was added. Immediately after this, the mixture was placed into a water bath kept at 70-80° C. and 100 mL of 10% (w/w) KNO₃ in water was added. The reaction mixture was stirred at this temperature for 60 to 90 minutes. After this time, the flask was removed from the water bath and the reaction mixture stirred for another 10 min. The resultant dark grey or black particles were collected by centrifugation, washed with deionised water, and lyophilized. The final product yield typically contains up to 70% (w/w) of iron in the form of Fe₃O₄, as determined by AAS or ESM, as shown in Table 4. The content of iron, as well as the recovery yield of the particles produced, can be regulated by the FeCl₂ concentration used in the formation of the chitosan-Fe(II) complex.

### EXAMPLE 3

### In Situ Synthesis of Superparamagnetic Sephadex Particles by the Nitrate Mediated Oxidation or Fe(II) Ions

A suspension of 5 g of Sephadex™ (epichlorohydrin cross-linked dextran beads, 20-40 μm in size) in 100 mL of fresh deionised water was added to 250 mL of an aqueous solution of 0.5 M FeCC₂. The suspension was stirred under reduced pressure, thereby removing all gases from the suspension and also facilitating diffusion of Fe ions into the porous Sephadex matrix. After 30 min of stirring, the swollen beads of the Sephadex-Fe complex were separated by centrifugation and washed several times with dejonised water. The resulting Sephadex-Fe particles were re-suspended in 250 mL of deionised water and 200 mL of 0.5 M NH₄OH was added. Immediately after NH₄OH addition, the mixture was placed into a water bath kept at 70°-80° C. and 30 mL of 10% (w/w) KNO₃ was added. The reaction mixture was stirred at this temperature for 80 min. After this time, the flask was removed from the water bath and the reaction mixture was stirred for another 10 min. The resulting particles were collected by centrifugation, washed with deionised water, washed with 0.1 M acetic acid, and lyophilized. The final product yield typically contains up to 15% (w/w) of iron in the form of Fe₃O₄, as determined by AAS or ESM, as shown in Table 4.

### EXAMPLE 4

### In Situ Synthesis of Superparamagnetic Cellulose Fiber Particles by the Nitrate Mediated Oxidation or Fe(II) Ions

Two pieces of cellulose sheets were weighed to give 0.75 g of the material (CLD fibers). The material was placed in 100 mL of deionised water to swell (15 min.) and the, hydrogel thus obtained transferred to a 10% (w/w) FeCl₂ solution in water (150 mL) to form a yellow mixture. After 40 min of stirring under vacuum, the cellulose-Fe complex were separated by centrifugation and washed several times with deionised water. The resulting Fe-cellulose fiber particles were re-suspended in 100 mL of deionised water and 200 mL of 0.5 M NH₄OH containing 1.0 g of KNO₃ was added. The reaction mixture was stirred at 70° C. for 40 min. The resulting magnetic fibers were collected by centrifugation, washed with dejonised water, washed with acetone, and dried in vacuo. The final product yield typically contains up to 35% (w/w) of iron in the form of Fe₃0₄, as determined by ESM, as shown in Table 4.

### EXAMPLE 5

### In Situ Synthesis of Superparamagnetic Chitin Crystallites by the Nitrate Mediated Oxidation or Fe(II) Ions

Chitin (10 g) was treated with a 5% (w/w) solution of sodium hypochlorite (250 mL) in water at 50° C. for one hour. The suspension was filtered and the oxidized chitin subsequently hydrolyzed in 100 mL of boiling 2.5 M HCl for 1 hour. The chitin suspension was then subjected to washing via centrifugation-dilution cycles with deionised water. When the pH approached 2.0-2.6, the material formed a colloidal suspension that could not be further separated from solution by slow-speed centrifugation. This suspension was then placed into dialysis tubes with a molecular weight cutoff limit of about 13-15 kDa and dialyzed against distilled water until a pH of 5.5-6.0 was achieved for the suspension.

The resulting suspension was added to 250 mL of an aqueous solution of 0.5 M FeCl₂. The suspension was stirred under reduced pressure, thereby removing all gases from the suspension and also facilitating diffusion of Fe ions into the chitin matrix. After 30 min of stirring, chitin-Fe complex was separated by centrifugation and washed several times with deionised water. The resulting chitin-Fe particles were re-suspended in 250 mL of deionised water and 200 mL of 0.5 M NH₄OH was added. Immediately after NH₄OH addition, the mixture was placed into a water bath kept at 70°-80° C. and 30 mL of 10% (w/w) KNO₃ was added. The reaction mixture was stirred at this temperature for 60 min. After this time, the flask was removed from the water bath and the reaction mixture was stirred for another 10 min. The resulting particles were collected by centrifugation, washed with deionised water, washed with 0.1 M acetic acid, and lyophilized.

### EXAMPLE 6

### In Situ Synthesis of Superparamagnetic Avicel® Particles by the Nitrate Mediated Oxidation of Fe(II) Ions

A suspension of 5 g of Avicel® (microcystalline cellulose) in 100 mL of fresh deionised water was added to 250 mL of an aqueous solution of 0.5 M FeCl₂. The suspension was stirred under reduced pressure, thereby removing all gases from the suspension and also facilitating diffusion of Fe ions into the Avicel® matrix. After 30 min of stirring, the Avicel®-Fe complex was separated by centrifugation and washed several times with deionised water. The resulting Avicel®-Fe particles were re-suspended in 250 mL of deionised water and 200 mL of 0.5 M NH₄OH was added. Immediately after NH₄OH addition, the mixture was placed into a water bath kept at 70°-80° C. and 30 mL of 10% (w/w) KNO₃ was added. The reaction mixture was stirred at this temperature for 60 min. After this time, the flask was removed from the water bath and the reaction mixture was stirred for another 10 min. The resulting particles were collected by centrifugation, washed with deionised water, washed with 0.1 M acetic acid, and lyophilized. The final product yield typically contains up to 10% (w/w) of iron in the form of Fe₃O₄, as determined by absorbance spectrometry (see Table 4).

### EXAMPLE 7

### TEMPO-Mediated Oxidation of MagCon Particles to Form MagCon-COOH

A suspension of 5 g of superparamagnetic cross-linked starch particles (MagCon particles) in 200 mL of deionised water was cooled to 2° C. TEMPO (2,2,6,6-tetramethylpiperidine-1-oxyl, 0.02 g) and NaBr (0.4g) were dissolved in 50 mL, of deionised water and then mixed with the MagCon suspension. The pH of this suspension was adjusted to 9.5 with 0.5M NaOH. A solution of sodium hypochlorite (60 mL, available chlorine 10-13%), also cooled to 2° C. and with the pH adjusted to 9.5 with 0.5M NaOH, was added to the MagCon suspension, keeping the reaction temperature at 20-5° C. The reaction mixture was stirred at 2°-5° C., while keeping the pH at 9.5 with 0.5M NaOH. After 30 minutes, the reaction was stopped by the addition of several drops of ethanol and the addition of 3M aqueous HCl to a pH of 7.0 or less. The resulting MagCon-COOH particles were separated by centrifugation, washed with deionised water several times, and lyophilized. Solid-state ¹³C-NMR spectra of MagCon and its carboxylated derivatives were recorded on a Chemganetics CMX-300 spectrometer. Integral intensities of the signals at 62 ppm (C-6 hydroxymethylene) and 178 ppm (C-6 carboxyl) (see FIG. 2) were used for determination of the degree of carboxylation (D.C.): D.C. (%)=[C-6 carboxyl/(C-6 hydroxymethylene+C-6 carboxyl)]×100. The degree of oxidation was determined to be in the range of 5-35%.

### EXAMPLE 8

### Synthesis of Amino-Containing of MagCon Particles

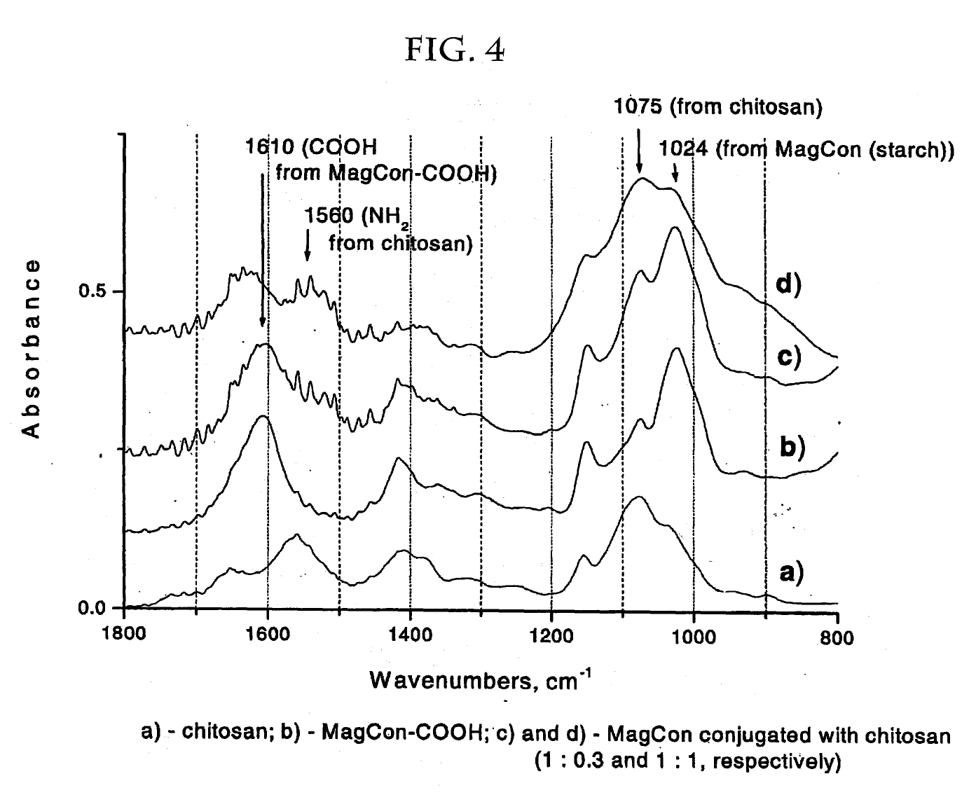

A suspension was formed with MagCon-COOH particles (1.0 g) in a 50 mL solution of chitosan (0.3 g to 1.0 g) in 50 mL of 0.1 M acetic acid. The mixture was rapidly stirred to produce a fine suspension. The pH of the suspension was raised to 10 with 0.1 M NaOH and the precipitated MagCon-COOH-chitosan salt was magnetically separated from the solution. The particles were washed with 0.05 M KH₂PO₄ buffer (pH, 5.0) and re-suspended in 50 mL of distilled water. To the stirred suspension was added EDC (1-[3-(dimethylamino)propyl]-3-ethylcarboduimide, 0.25 g) and stirring was continued for 3 hours. The resulting MagCon-Chitosan particles were separated magnetically, washed with deionised water, and lyophilized. FTIR spectra of chitosan (a), MagCon-COOH (b), MagCon-COOH conjugated with chitosan, 1:0.3 (c), and MagCon-COOH conjugated with chitosan, 1:1 (d) are shown in FIG. 4.

### EXAMPLE 9

### Attachment of Bovine Serum Albumin (BSA) to MagCon Particles

Following is a general procedure that can be used to couple proteins, or any other biological molecule containing free primary amines, to MagCon particles that have been formed via the nitrate oxidation of Fe(II).

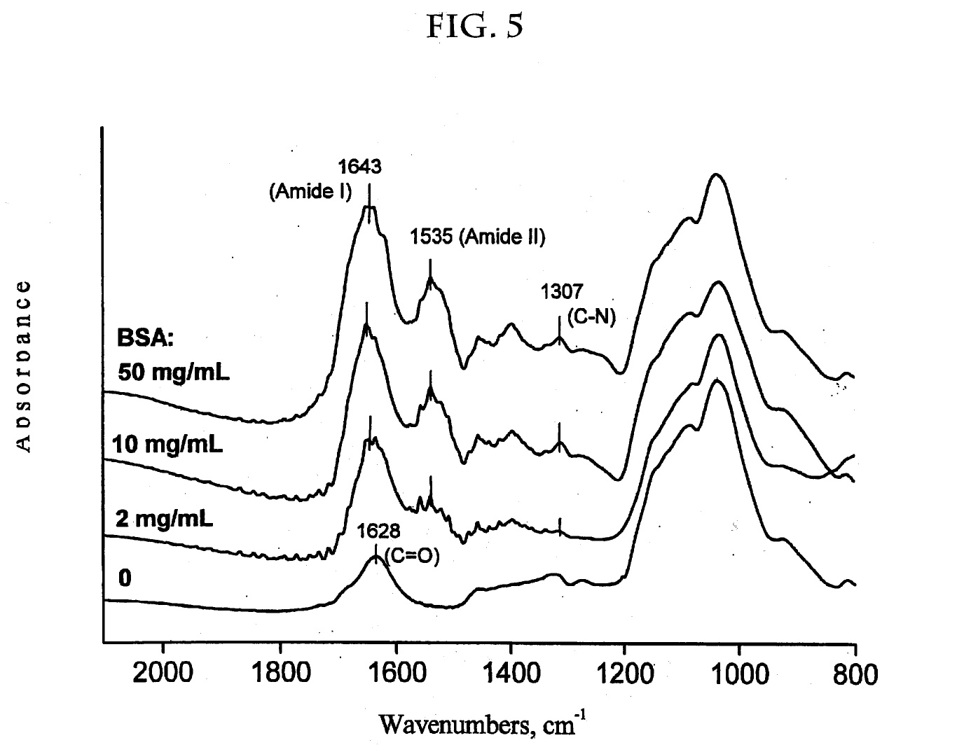

A 0.01 M phosphate buffer in 0.15 M NaCl was prepared by dissolving KH₂PO₄ (1.74 g) and NaCl (8.7 g) in 800 mL of water and adjusting the pH of this buffer solution to 6.8-7.2 with phosphoric acid, followed by adjusting the volume of the solution to 1.0 L. MagCon particles (25 mg) were suspended in 5 mL of the phosphate buffer and the suspension was mixed for 30 minutes to allow the particles to swell. The particles were separated magnetically and the supernatant was removed. The particles were suspended in and the separated from phosphate buffer three additional times. To the particles were added 5 mL of a 50 mM sodium periodate solution (1.08 g of NaIO₄ in 100 mL of H₂O) and the suspension was shaken well and incubated for 30 min. at room temperature. The particles were separated magnetically and the supernatant removed to produce activated MagCon particles. The particles were then suspended in and magnetically separated from phosphate buffer three times. A solution of bovine serum albumin (BSA) was prepared by dissolving 5-50 mg of the protein in 1 mL of phosphate buffer and this solution was transferred to a tube containing the activated MagCon particles from above. The tube was shaken and incubated for 2-3 hours at room temperature. A fixation solution containing 1 g of sodium cyanoborohydride in 100 mL of deionised water was prepared and 0.25 mL was immediately added to the mixture of MagCon particles reacted with protein. The tube was well shaken for 30 minutes, followed by magnetically separating the particles and removing the unreacted protein solution. A quenching solution was prepared by dissolving 7.5 g of glycine in 90 mL of deionised water, adjusting the pH to 8.0 with 1.0 M NaOH, and adjusting the volume of the solution to 100 mL with dejonised water. To the MagCon-protein particles formed above was added 5 mL of the glycine quenching solution and 0.5 mL of the cyanoborohydride fixation solution. The suspension was mixed well for 1 hour and the particles magnetically separated. A wash buffer was prepared by dissolving 1.21 g of Tris buffer, 1.0 g of sodium azide, 8.7 g of NaCl, and 0.37 g of EDTA in 800 mL of deionised water. The pH of the wash buffer was adjusted to 7.0-7.2 with 0.1 M HCl and the volume adjusted to 1.0 L. The magnetic particles were treated with the wash buffer four times. Each time the particles were well shaken with the buffer followed by magnetic separation and removal of the buffer. After this wash sequence, the protein-bound MagCon particles were ready for use. FIG. 5 is a composite of FTIR spectra of MagCon conjugated to bovine serum albumin (BSA) at BSA reaction mixture concentrations of 0 mg/mL, 2 mg/mL, 10 mg/mL, and 50 mg/mL. Amide N—H and C—N vibrations were observed in the FTIR of the protein-conjugated beads, as shown in FIG. 5.

### EXAMPLE 10

### Attachment of Bovine Serum Albumin (BSA) to MagChi

Following is a general procedure that can be used to crosslink proteins, or any other biological molecule containing free primary amines, to MagChi particles, or any other superparamagnetic particles containing a primary amine, which have been formed via the nitrate oxidation of Fe(II).

A 0.01 M phosphate buffer in 0.15 M NaCl was prepared by dissolving KH₂PO₄ (1.74 g) and NaCl (8.7 g) in 800 mL of deionised water and adjusting the pH of this buffer solution to 6.8-7.2 with phosphoric acid, followed by adjusting the volume of the solution to 1.0 L. MagChi particles (25 mg) were suspended in 5 mL of the phosphate buffer and the suspension was mixed for 30 minutes to allow the particles to swell. The particles were separated magnetically and the supernatant was removed. The particles were suspended in and the separated from phosphate buffer three additional times. To the particles were added 5 mL of a 5% (v/v) solution of glutaraldehyde in the phosphate buffer prepared above and the suspension was shaken well and incubated for 30 min. at room temperature. The particles were separated magnetically and the supernatant removed to produce activated MagChi particles. The particles were then suspended in and magnetically separated from phosphate buffer three times. A solution of bovine serum albumin (BSA) was prepared by dissolving 5-50 mg of the protein in 1 mL of phosphate buffer and this solution was transferred to a tube containing the activated MagChi particles from above. The tube was shaken and incubated for 3-5 hours at room temperature. A fixation solution containing 1 g of sodium cyanoborohydride in 100 mL of delonised water was prepared and 0.25 mL was immediately added to the mixture of MagChi particles that had been treated with protein. The tube was well shaken for 30 minutes, followed by magnetically separating the particles and removing the unreacted protein solution. A quenching solution was prepared by dissolving 7.5 g of glycine in 90 mL of deionised water, adjusting the pH to 8.0 with 1.0 M NaOH, and adjusting the volume of the solution to 100 mL with deionised water. To the MagChi-protein particles formed above was added 5 mL of the glycine quenching solution and 0.5 mL of the cyanoborohydride fixation solution. The suspension was mixed well for 1 hour and the particles magnetically separated. A wash buffer was prepared by dissolving 1.21 g of Tris buffer, 1.0 g of sodium azide, 8.7 g of NaCl, and 0.37 g of EDTA in 800 mL of deionised water. The pH of the wash buffer was adjusted to 7.0-7.2 with 0.1 M HCl and the volume adjusted to 1.0 L. The magnetic particles were treated with the wash buffer four times. Each time the particles were well shaken with the buffer followed by magnetic separation and removal of the buffer. After this wash sequence, the protein-bound MagChi particles were ready for use. A comparison of the amount of protein conjugated to MagCon and MagChi particles is shown in Table 5.

TABLE 5
Amount of bovine serum albumin (BSA) conjugated to MagCon and
MagChi particles, as determined by mass balance.

| Reaction | Ratio of BSA conjugated to | Ratio of BSA conjugated |
|---|---|---|
| concentration of | MagChi (mg of protein/mg | to MagCon (mg of |
| BSA (mg/mL) | of particle) | protein/mg of particle) |
| 1.0 | 0.000 | 0.002 |
| 2.0 | 0.002 | 0.023 |
| 3.0 | 0.010 | 0.033 |
| 5.0 | 0.027 | 0.055 |
| 7.5 | 0.048 | 0.082 |
| 10 | 0.057 | 0.110 |
| 25 | 0.093 | 0.176 |
| 50 | 0.013 | 0.221 |

### EXAMPLE 11

### Attachment of Bovine Serum Albumin (BSA) to MagCon-COOH

Following is a general procedure that can be used to crosslink proteins, or any other biological molecule containing free primary amines, to MagCon-COOH particles (2.0% COOH, 5.0% COOH, 10.0% COOH, 25.0% COOH, 50% COOH were used) or any other superparamagnetic particles containing a carboxyl group, which have been formed via the nitrate oxidation of Fe(II).

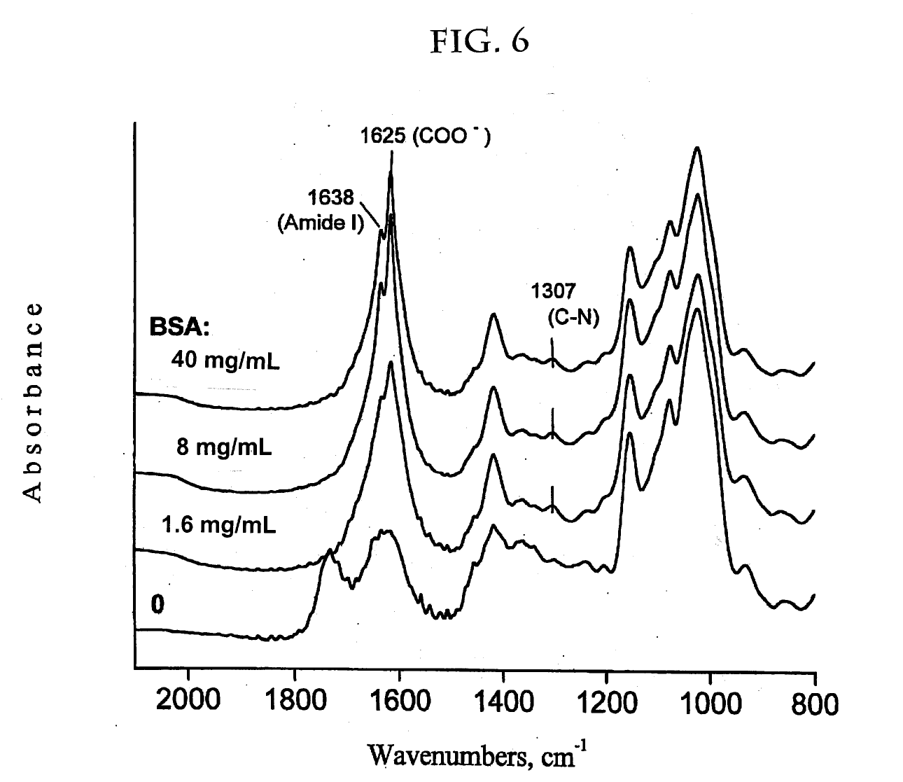

A 0.01 M phosphate buffer in 0.15 M NaCl was prepared by dissolving KH₂PO₄ (1.74 g) and NaCl (8.7 g) in 800 mL of deionised water and adjusting the pH of this buffer solution to 6.8-7.2 with phosphoric acid, followed by adjusting the volume of the solution to 1.0 L. MagCon-COOH particles (25 mg) were suspended in 5 mL of the phosphate buffer and the suspension was mixed for 30 minutes to allow the particles to swell. The particles were separated magnetically and the supernatant was removed. The particles were suspended in and the separated from phosphate buffer three additional times. A solution of bovine serum albumin (BSA) was prepared by dissolving 5-50 mg of the protein in 1 mL of phosphate buffer and this solution was transferred to a tube containing the MagCon-COOH particles from above. To the particles were added 0.25 mL of a 0.2% (w/w) solution of 1-[3-(dimethylamino)propyl]-3-ethylcarbodiimide (EDCI) in water. The suspension was shaken well and incubated for 2-3 hours at room temperature. The particles were separated magnetically and the supernatant removed to produce MagCon-COOH protein particles. A quenching solution was prepared by dissolving 7.5 g of glycine in 90 mL of delonised water, adjusting the pH to 8.0 with 1.0 M NaOH, and adjusting the volume of the solution to 100 mL with deionised water. To the MagCon-COOH-protein particles formed above was added 5 mL of the glycine quenching solution. The suspension was mixed well for 1 hour and the particles magnetically separated. A wash buffer was prepared by dissolving 1.21 g of Tris buffer, 1.0 g of sodium azide, 8.7 g of NaCl, and 0.37 g of EDTA in 800 mL of deionised water. The pH of the wash buffer was adjusted to 7.0-7.2 with 0.1 M HCl and the volume adjusted to 1.0 L. The magnetic particles were treated with the wash buffer four times. Each time the particles were well shaken with the buffer followed by magnetic separation and removal of the buffer. After this wash sequence, the protein-bound MagCon-COOH particles were ready for use. A comparison of the ratio of BSA incorporation to MagCon-COOH particles of differing carboxylation percentages is shown in Table 6. Amide N—H and C—N vibrations were observed in the FTIR of the protein-conjugated beads, as shown in FIG. 6.

TABLE 6
Amount of bovine serum albumin (BSA) conjugated to
MagCon - COOH, as determined by mass balance.

| | Ratio of BSA | Ratio of BSA | Ratio of BSA |
|---|---|---|---|
| | conjugated to | conjugated to | conjugated to |
| Reaction | MagCon - 8.5% | MagCon - 12% | MagCon - 30% |
| of | COOH (mg of | COOH (mg of | COOH (mg of |
| concentration | protein/mg of | protein/mg of | protein/mg of |
| BSA (mg/mL) | particle) | particle) | particle) |
| 1.6 | 0.0002 | 0.0021 | n/d |
| 4.0 | 0.215 | 0.051 | 0.054 |
| 8.0 | 0.051 | 0.085 | 0.149 |
| 20.0 | 0.099 | 0.132 | 0.235 |
| 40.0 | 0.131 | 0.166 | 0.371 |

From the foregoing description, it will be apparent that variations and modifications may be made to the invention described herein to adopt it to various usages and conditions. Such embodiments are also within the scope of the following claims.

All publications mentioned in this specification are herein incorporated by reference to the same extent as if each independent publication or patent application was specifically and individually indicated to be incorporated by reference.

What is claimed is:

1. A method for the in situ formation of superparamagnetic particles in a polysaccharide matrix, said method comprising:
(a) diffusing an Fe(II) salt into said starch matrix, thereby entrapping Fe(II) ions within the matrix; and
(b) oxidizing said entrapped Fe(II) ions with nitrate under alkaline conditions to convert said Fe(II) ions into superparamagnetic ferric oxide particles.

2. The method of claim 1, wherein said polysaccharide is starch, cross-linked starch, chitosan, chitin crystallites, dextran, cross-linked dextran, cellulose, cellulose fibers, microcrystalline cellulose, alginic acid, hyaluronic acid, glycogen, or a glycosylaminoglycan.

3. The method of claim 2, wherein said polysaccharide is cross-linked starch, cross-linked dextran, chitosan, chitin crystallites, microcrystalline cellulose, or cellulose fibers.

4. The method of claim 3, wherein said polysaccharide is cross-linked starch.

5. The method of claim 3, wherein said polysaccharide is chitosan.

6. The method of claim 1, wherein said alkaline conditions are provided by contacting said matrix with ammonium hydroxide.

7. The method of claim 1, wherein said nitrate is sodium nitrate, potassium nitrate, cesium nitrate, ammonium nitrate, tetra(C₁-C₈ alkyl)ammonium nitrate, silver nitrate, or barium nitrate.

8. The method of claim 7, wherein said nitrate is potassium nitrate.

9. The method of claim 1, wherein FeCl₂ is used as a source of Fe(II) ions.

10. A method for the in situ oxidation of a superparamagnetic iron particle polysaccharide matrix comprising the following steps:
a) preparing a solution or a suspension of said superparamagnetic iron particle matrix
b) adding sodium bromide and a catalytic amount of TEMPO (2,2,6,6-tetramethyl-1-piperidinyloxy, free radical) to said solution or suspension while maintaining the solution/suspension of said superparamagnetic iron particle matrix at a temperature of about 0° C. to about 20° C. and a pH of between about 7.5 to about 10.5 pH units; and
c) adding sodium hypochlorite to the solution or suspension while maintaining the solution/suspension of said superparamagnetic iron particle matrix at a temperature of about 0° C. to about 20° C. and a pH of between about 7.5 to about 10.5 pH units.

11. The method of claim 10, comprising the addition of sodium chlorite after step c).

12. The method of claims 10 or 11, wherein said superparamagnetic particle is formed by the method of claim 1.

13. A composition prepared by the method of any of the claims 10 to 12 comprising:
(a) a starch-based matrix; and
(b) a superparamagnetic iron oxide particle within said matrix,
wherein said composition comprises from about 5 percent to about 30 percent mole percent COOH groups.

14. A composition prepared by the method of claim 1 or 12 comprising:
(a) a polysaccharide matrix; and
(b) a superparamagnetic iron oxide particle within said matrix.

15. The composition of claim 14 comprising a biological molecule other than said polysaccharide matrix.

16. The composition of claim 15, wherein said biological molecule is covalently attached to said polysaccharide matrix.

17. A method for the separation, isolation, identification, or purification of a biological entity, said method comprising:
a) contacting a sample containing said biological entity with a composition of any of the claims 13 to 16.
b) affecting the separation, isolation, identification, or purification of said biological entity by the application of a magnetic field.

18. The method of claim 17, wherein said composition comprises a biological molecule that interacts positively with said biological entity.

19. The method of claim 18, wherein said biological entity comprises a cell, a virus, or a phage.

20. The method of claim 18, wherein said biological entity comprises a protein, a peptide, a carbohydrate, a glycopeptide, a glycoprotein, a glycosylaminoglycan, a cationic lipid, a glycolipid, or a polynucleotide.

21. The method of claim 20, wherein said biological entity comprises a protein.
